(19) United States
(12) Patent Application Publication
(10) Pub. No.: US 2021/0190777 A1
(43) Pub. Date: Jun. 24, 2021

(54) ANALYZING DEVICE HAVING FUNCTIONALIZED CRYOGELS

(71) Applicant: Albert-Ludwigs-Universitat Freiburg, Freiburg (DE)

(72) Inventors: Marc Zinggeler, Aesch (CH); Jurgen Ruehe, Eichstetten (DE); Thomas Brandstetter, Freiburg (DE)

(73) Assignee: Albert-Ludwigs-Universitat Freiburg, Freiburg (DE)

(21) Appl. No.: 16/471,897

(22) PCT Filed: Dec. 20, 2017
(86) PCT No.: PCT/EP2017/083825

§ 371 Date: May 18, 2020

(30) Foreign Application Priority Data
Dec. 20, 2016 (DE) 10 2016 015 171.4

(51) Int. Cl. G01N 33/543; G01N 21/64; B01L 3/00

(52) U.S. Cl. CPC G01N 33/54386; G01N 21/6428; B01L 3/502715; G01N 33/54353; B01L 2300/0816; B01L 2300/123; B01L 2300/168; B01L 2300/0627; B01L 2200/10; B01L 2300/069; B01L 2300/12; B01L 2400/0406

(57) ABSTRACT

A device having a channel with a sequence of compartments having molecules having specific binding sites, this sequence of compartments being suitable for the specific binding of analytes, characterized in that the molecules having specific binding sites are bound to porous cryogels as carriers and the cryogels are chemically bonded to the wall of the channel, a method for producing the device and the use of the device in analytics.

### BACKGROUND

The invention relates to a device comprising a channel having a sequence of compartments containing molecules having specific binding sites, which sequence of compartments is suitable for the specific binding of analytes.

A multiplicity of analytical methods makes use of the specific binding of an analyte to a molecule immobilized on a solid substrate surface and having a specific binding site, for example a receptor. The bound sample molecule is then generally detected and quantified by direct or indirect optical methods, such as staining or fluorescent labeling, followed by a colorimetric or fluorescence-spectroscopic evaluation.

Technological platform methods which make it possible to carry out such assays in a rapid, cost-effective, multiparametric and quantitative manner with high sensitivity and large dynamic measurement range are, then, of utmost interest.

Typically, rapid test platforms use porous materials which have been functionalized at differentiable sites and through which the sample to be analyzed then flows. In the classic lateral flow tests (LFTs), the porous support is a nitrocellulose membrane. Weaknesses of LFTs are found in the low sensitivity and precision thereof and the greatly limited measurement range.

WO 03/029480 describes the measurement of analytes using cryogels, wherein the cryogels are provided as blocks and cut into slices in order to present molecules which were introduced zonally into the cryogels and serve for the detection of analytes.

WO 2008/145722 describes conventional separation columns which have been filled with different porous materials in layered cylindrical segments separated from one another by reflective separation elements, with binding sites for affinity-chromatographic evaluation by means of light transmittance measurement that are different for each segment being provided in each case.

X. Qu et al., Biosensors and Bioelectronics 38, 2012, 342-347, describes a method for determining protein inclusion bodies (which, for example, can be found when producing proteins in genetically modified microorganisms) by means of an enzyme-linked immunosorbent assay (ELISA) using macroporous cryogel miniplates/minicolumns. Use of capillaries or binding to a column wall or a sequential sequence of different binding sites are not described.

J. Ahlqvist et al., Analytical Biochemistry 354, 2006, 229-237, describes capillaries, on the alkoxyaminosilane- and glutaraldehyde-pretreated wall of which different sample molecules, which react with the pretreated wall for 1 hour to 24 hours, are bound covalently in sections by sequential loading with different solutions, which sample molecules can then be detected by means of binding of detector molecules, for example by fluorescence measurement. Filling as well with sequentially interposed pure carrier liquids (without binding molecules) for better separation of different molecules to be detected is mentioned. Porous support materials are not mentioned.

### SUMMARY

Against this background, it is an object of the invention to minimize weaknesses like the ones described (especially for LFTs) and to avoid a complicated production process (prior production and functionalization of the filter elements, construction with reflective separation elements to reduce radiation overlap, only limited potential for miniaturization).

Against this background, the invention describes the production and use of spatially separated, linearly arranged, functional cryogels in a transparent support as an assay platform.

In a first aspect, the invention therefore provides a device as stated at the beginning, which is characterized in that the molecules having specific binding sites are bound to porous cryogels as support and the cryogels are chemically bonded to the wall of the channel.

The invention also provides a process for producing such a device, characterized in that, alternately, volumes of initially charged solutions containing, firstly, precursor molecules of porous cryogels as support and, secondly, molecules having specific binding sites for the specific binding of analytes that are to be immobilized and are different for each volume, or the precursor molecules thereof, are supplied in sequence per channel, it being possible if desired to supply—between volumes containing the cryogel precursors and the molecules having specific binding sites that are to be immobilized or the precursor molecules thereof—additionally precursor molecules of the cryogels without molecules to be immobilized or other liquid or gaseous separation substances in order to ensure a clearer separation of the cryogels containing different molecules as specific binding sites; the filled device is cooled in order to freeze the solutions contained therein; and then the reactions to develop the formation of the cryogels, the binding thereof to the wall of the channel and the binding of the molecules having specific binding sites that are to be immobilized or the precursor molecules thereof are carried out; and, preferably, the cryogel is thawed and rinsed.

In a third aspect, the invention provides for the use of a device according to the invention, in which a liquid or gaseous sample containing analytes is conducted through a device of the stated kind and bound molecules are identified and preferably also quantified.

Instead of the more general terms above and below, one or more thereof may be replaced with one or more of the following more specific definitions, leading in each case to preferred embodiments of the invention:

What is especially possible as the device are microfluidic elements, such as microfluidic chips (microchips containing microfluidic channels or hereinafter “microchannels”) or particularly capillaries, especially those made of plastic or of glass.

Microfluidic elements are preferably those systems, including capillaries, that comprise one or more channels, i.e., passages, chambers or lines, which have at least one (1) internal cross-sectional dimension, e.g., depth, width, length, or especially (smallest) diameter transverse to the longitudinal direction across parts or preferably the entire length of the microchannel, on the μm scale, preferably of 1000 or 800 μm or less, especially in the range from 0.1 to 750 μm, for example between 5 and 500 μm. The microfluidic elements according to the invention contain at least one channel on the μm scale (microchannel) as specified or several thereof, which and can be present in a very wide variety of different shapes or geometries, for example straight, sawtooth-shaped, branched, meander-shaped, spiral, circular or the like.

What are preferred as microfluidic element are one or more capillaries which each contain a microchannel as defined above.

A range of materials can be used for the capillaries or the microfluidic chips. Preferably, when the devices are produced microtechnically, the materials are chosen such that they are compatible with known microfabrication techniques, for example photolithography, chemical wet etching, plasma or laser ablation, injection molding or other methods based on original molds, pressing, embossing, CVD coating technique and the like. In general, silicon-based materials, such as silicon, silicon dioxide, glass, quartz, silicone or polysilicone, or other semiconductor materials, such as gallium arsenide, are especially mentionable. Further preferred materials are, inter alia, plastics (polymers), such as polymethyl methacrylate (PMMA), polycarbonate, polyester, polyamides, polytetrafluorethylene (TEFLON®), polyvinyl chloride (PVC), polydimethylsiloxane (PDMS), polysulfone, polystyrene, polyolefins, such as polymethylpentene, polypropylene or polyethylene, polyvinylidene fluoride, acrylonitrile-butadiene copolymer (ABS), block copolymers or mixtures of two or more thereof. Such polymeric microfluidic chips, for example comprised of a substrate layer and a cover layer, are producible by known microfabrication techniques, for example the abovementioned microfabrication techniques, or by microtechnically produced mother molds using known molding methods, such as injection molding, embossing or stamping, or by polymerization of the monomeric precursor material within the mold (original mold). Such polymeric materials are preferred, since they can be easily producible, inexpensive and also disposable. However, polymer- or glass-based capillaries are particularly preferred. All the stated materials can contain specifically treated or coated surfaces or surface sections (e.g., within the microchannels), for example by silanization (coating with a silane to which a connecting molecule bearing a hydroxyl group is coupled), etching of glass so that the surface has many hydroxyl groups, for example using Caro's acid, or some other introduction of reactive groups (e.g., epoxy groups, activated ester groups, diene or dienophilic groups or the like).

The microchannels and/or microchambers of the microfluidic elements are produced especially with use of the customary microfabrication techniques.

Preferably, one surface of a cover layer and the side of a substrate layer comprising microchannels and/or microchambers are joined to one another, for example by clamping, adhesive bonding or fusion, with the result that the cover layer upwardly completes and seals off the channels or chambers. Outwardly leading supply and discharge elements for liquids are guided through the cover layer or at the side or bottom of the substrate layer.

In the case of capillaries, these can be obtained by stretching of tubes comprised of a stretchable (at least with heat) material (such as a thermoplastic or glass). Examples of suitable capillaries are those which are commercially available under the name Minicaps from Hirschmann Laborgeräte GmbH & Co. KG, Hauptstraße 7-15, 74246 Eberstadt, Germany.

Preferably, the entire microfluidic element is optically transparent in each case, i.e., especially capable of allowing the passage of UV, visible or infrared light. For example, quartz or glass or transparent polymeric materials, such as PMMA or polycarbonate, are suitable optically transparent materials.

A channel is thus especially a microchannel as described above, preferably having a diameter as specified as preferred above.

Specific binding of analytes (these are, for example, cells, cellular constituents or fractions, viruses, viral fragments or especially molecules in a medium to be analyzed) is especially to be understood to mean binding based on a specific interaction such as that between enzyme substrate and active site of an enzyme, a receptor and its molecule to be bound, oligonucleic or polynucleic acids upon (especially stringent) hybridization, and especially antigen-and-antibody, biotin-avidin or biotin-streptavidin binding, with covalent bonds also being conceivable (e.g., such as in the case of proteinase inhibitors which bind covalently). In connection with this, the analytes can, for example, be low-molecular-weight organic compounds, such as drug substances, lipids, mono- or oligosaccharides, amino acids, mono- or oligopeptides, mono- or oligonucleotides or plant protectants; nucleic acids; proteins; polysaccharides; glycoproteins; or other naturally or otherwise occurring organic molecules, cells, cellular constituents or fragments (e.g., organelles such as mitochondria, plastids or the like), viruses, viral fragments, molecules (including molecular complexes).

In connection with this, molecules having specific binding sites can, for example, be enzymes, receptors, antigens, antibodies or fragments thereof having the specific binding site of corresponding antibodies, other proteins, peptides, biotin, avidin, streptavidin, aptamers, MIP (molecularly imprinted polymer) or sequence-specific oligonucleotides or polynucleotides.

Cryogels are (micro)porous polymer networks which are produced by polymerization reactions or by crosslinking of polymeric precursors in moderately frozen solutions. In this connection, moderately frozen means that microphase separation takes place and systems consisting of crystallized solvent (typically water-based) and a “liquid microphase” (LMP), a non-frozen proportion of the concentrated precursor solutions, are obtained. In such systems, the reactions leading to the shaping of the polymer networks take place only in the liquid phase. As the reaction advances, this phase becomes more viscous and ultimately solid. After the solvent crystal regions are thawed, what remains is a network of pores connected to one another.

What are especially possible as porous cryogels as support for the bound molecules having specific binding sites are those based on monomers or comonomers, or prepolymers, as precursor molecules, which can polymerize by addition reaction and/or free-radical reaction. Examples are isocyanates, which can react with polyamines to form polyurethanes, epoxides, which can react with polyamines or polyols to form corresponding polyadducts, crosslinking of amino group-containing molecules (especially polymers) with bifunctional aldehydes (e.g., glutaraldehyde) or especially monomers, comonomers, prepolymers or else polymers which can be crosslinked by irradiation with visible or ultraviolet light. Examples thereof are monomer molecules having ethylenically unsaturated, free-radically curable (e.g., vinyl) groups, such as acrylamide, N,N-methylenebis(acrylamide), allyl glycidyl ether, vinyl ester or vinyl urethane precursors, or monomer mixtures of two or more thereof for copolymers, or prepolymers of the stated compounds, OCT (e.g., CryoGel OCT from Instrumedics Inc., Hackensack, N.J.) from WO 03/029480 or furthermore azides, such as aryl azides or azidomethyl coumarins, benzophenones, anthraquinones, certain diazo compounds, diazinines, psoralens (psoralen derivates) or the like, especially copolymers which preferably contain functional groups such as vinyl, epoxy or aldehyde groups or especially benzophenone groups. In this connection, the porosity arises through freezing of the particular solvent, yielding “solvent crystals” (e.g., ice crystals) around which the polymers form during the reaction (e.g., under irradiation with UV or visible light), with melting and thus removal of the thawed solvent giving rise to the cavities of the thus porous (spongy) cryogel.

In general, a multiplicity of methods can be theoretically envisaged. What is important is that the cryogelling, the immobilization of the molecules having a specific binding site and the attachment to the wall take place together.

The chemical bond between cryogel and the wall of the channel is primarily based on direct binding or furthermore binding via a bridge former, generated or generable in both cases by chemical reaction, especially upon irradiation with gamma radiation, electron radiation or especially UV light or visible light.

Chemically bonded means that there is no dissociation from the surface (except in the case of an intended detachment) of the wall of the channel, for example microchannel, with the customary steps for washing and reagent supply.

The use of a device according to the invention as well as the production thereof can be effected with appropriate mechanisms for controlling the flows and residence times of solutions, such as buffers, reagents, enzymes, enzyme substrates, etc., such as means for generating pressure differences, for example centrifuges or other centrifugal force-generating devices, external upstream or downstream pump systems (pump devices), and/or absorption materials, such as superabsorbent polymers, which can supply or discharge liquids in a reproducible manner, in reproducible and accurately controlled amounts, and which also maintain the purity and, if desired, the sterility of solutions. The 205U multichannel cassette pump from Watson Marley, Inc. (USA) is an example of such a pump. Alternatively, miniaturized mechanical pumps, based on microelectromechanical systems (MEMS), can be used. These miniature pumps can be internal or external in relation to microchannels and reaction chambers or the like. An example of such pumps are the ones described by Shoji et al. in Electronics and Communications in Japan, Part 2, 70, 52-59 (1989), or diaphragm pumps, thermal pumps or the like. Examples are especially peristaltic pumps, such as a tubing pump (e.g., IPC ISMATEC from Cole-Parmer GmbH, Futtererstr. 16, 97877 Wertheim, Germany) which is equipped with suitable tubing (e.g., with ID 0.51 mm).

Other suitable means for conveying liquids through (micro)channels encompass electrokinetic pumps—based on the principle of electroosmosis—for example as described by Dasgupta et al. in Anal. Chem. 66, 1792-1798 (1994), or electrophoretic methods, which require, for example, the use of inert metallic electrodes (e.g., made of gold or platinum) which are in contact with external or internal circuits or electrical connections and suitable controllers (see, for example, U.S. Pat. No. 5,858,195).

The inflow and outflow can also be regulated by microvalves which function on the principle of piezoelectric bending, for example with polysilicone- or lithium niobate-based piezo elements, on the basis of ultrasonic sensors, on the basis of tongue-shaped elements which are bent under the influence of an external magnetic field due to magnetostrictive forces, of slide-controlled valves or of fluidic elements in which small liquid movements control large, and the like. Such elements are, for example, described in U.S. Pat. No. 5,837,200.

Electrical controllers as well are preferably present during use, for example for controlling pumps, light elements and the like.

In a preferred variant, the flow is achieved solely or at least in part by means of the capillary forces acting in microfluidic channels. Preferably, in this connection, the necessary movement of liquid, especially the flow-through of liquid amounts exceeding the microfluidic channel volume, can be caused or assisted by coupling to an absorbent material, such as especially a superabsorbent material, downstream of the microfluidic channels or placed at the end thereof.

For the production, one or more of the following definitions are preferably applicable:

In volumes (liquid portions=liquid compartments) of initially charged solutions which contain precursor molecules of porous cryogels as support and which, on the other hand, contain molecules having specific binding sites for the specific binding of analytes that are to be immobilized and are different for each volume (liquid portion), or the precursor molecules thereof, the initially charged solutions can, for example, be aqueous buffer solutions, such as Michaelis barbital/acetate buffer (pH 2.6 to 9.2), acetic acid/acetate buffer (pH 3.7 to 5.7), Good's buffers, including for example HEPES: 4-(2-hydroxyethyl)-1-piperazineethanesulfonic acid (pH 6.8 to 8.2), HEPPS: 4-(2-hydroxyethyl)piperazine-1-propanesulfonic acid (pH 7.3 to 8.7) or MES: 2-(N-morpholino)ethanesulfonic acid (pH 5.2 to 6.7), carbonic acid/silicate buffer (pH 5.0 to 6.2; mildly acidic), phosphate buffer: NaH₂PO₄+Na₂HPO₄ (pH 5.4 to 8.0), optionally supplemented with sodium chloride (phosphate-buffered saline=PBS), carbonic acid/bicarbonate buffer (pH 6.2 to 8.6; neutral), TRIS: tris(hydroxymethyl)aminomethane (pH 7.2 to 9.0), ammonia buffer: NH₃+H₂O+NH₄Cl (pH 8.2 to 10.2) or citric acid or citrate buffer, without or additionally, for example, with detergents dissolved therein, such as polysorbates ((polyoxyethylene sorbate) 20, 21, 40, 60, 61, 65, 80, 81, 85, 120, for example Tween®).

Separation substances can be corresponding solutions without the precursor molecules of the cryogels, or other liquids, including for example water-immiscible liquids, such as hydrocarbons (linear, branched, cyclic, saturated or completely or partially unsaturated) having 3 to 40 carbon atoms, for example pentane or hexane, superfluid CO₂ or gases. They allow a clear spatial separation of cryogel sections in the channel containing different immobilized molecules having different specific binding sites. However, they can also be omitted in the production, with the result that slight mixing effects may occur in edge regions owing to the different solution portions—the measurements then preferably take place in regions without mixing.

In this connection, sequential supplying is preferably achieved by the above-described control of the flows and residence times of solutions using the stated devices.

The cooling of a filled device in order to freeze the solutions contained therein is preferably achieved by means of a cold gas, for example cold air, a cooling bath, for example liquid air or liquid nitrogen, at temperatures below the melting point of the respective solutions, for example in the range from 0° C. to minus 80° C., for example from −10° C. to −30° C.

The reactions to develop the formation of the cryogels, the binding thereof to the wall of the channel and the binding of the molecules having specific binding sites that are to be immobilized or the precursor molecules thereof are preferably carried out by irradiation with UV and/or visible light in order to achieve the polymerization and/or (especially in the case of (pre)polymers that are used) crosslinking (especially as free-radical crosslinking of the cryogel precursors, the attachment of the molecules to be immobilized and the attachment of the cryogel matrix to the wall of the channel(s).

For this purpose, suitable radiation sources, such as lasers, halogen lamps, incandescent lamps, plasma lamps, LEDs or the like, are used. The radiation dose (results from intensity and duration of irradiation) is selected such that a sufficient reaction is ensured, for example in the range from 0.1 to 500, such as from 1 to 50, J/cm².

Lastly, after thawing, the channel(s) of the device obtained can preferably be rinsed with a rinse solution (which also nonspecific binding site-saturating compounds, such as serum albumin, for example bovine serum albumin, for example with an above-described solution without molecules to be immobilized or with one of the separation liquids.

The use of a device according to the invention, in which a liquid or gaseous sample containing sample molecules is conducted through a device of the stated kind and bound molecules are identified and preferably also quantified, takes place under customary conditions allowing specific (generally noncovalent) binding of the sample molecules (analytes), as described above for the specific binding of analytes.

Examples of possible biological samples containing analytes to be tested are: cell suspensions (e.g., animal cells, plant cells, protistic cells, bacterial cells, fungal cells or mixtures thereof), water samples from bodies of water, such as puddles, ponds, lakes, streams, rivers or the sea, groundwater, biological liquids such as sap, blood, blood products, urine, sweat, tears, saliva, amniocentesis samples, sperm, mucosa, or the like.

The optically (including for UV light) transparent regions of the device according to the invention are especially suitable for monitoring and control. Suitable detection systems for this purpose are, for example, those which can capture colorimetric, fluorometric or radioactive signals or chemiluminescent signals, or additionally changes in the refractive index or the density, for example by means of photoacoustic units. Examples of suitable detectors are spectrophotometers, photodiodes, photomultipliers, microscopes, scintillation counters, cameras, cooled charge-coupled device cameras (CCD), films and the like. Optical detection systems are especially preferred; for instance, fluorescence-based signals are for example measured by means of laser-activated fluorescence detection systems using lasers having a suitable wavelength in order to activate the fluorescence indicator within the system. The fluorescence is then for example measured by means of a photomultiplier tube. For colorimetric detection, spectrophotometric detection systems which detect a light source directed toward the sample and allow a measurement of the absorbance or the optical transparency are preferably used (cf. The Photonics Design and Applications Handbook, books 1, 2, 3 and 4, yearly from Laurin Publishing Co., Berkshire Common, Pittsfield, Mass., USA with sources of optical components).

Nonoptical detection systems can be used too, for example temperature sensors (useful for endothermic or exothermic reactions), conductivity, impedance (e.g., by ISFETS), potentiometry (pH, ions) or amperometry (when using oxidizable or reducible reagents, such as oxygen or organic oxidizable or reducible reagents). Examples of useful detection systems are immunological systems, for example those allowing detection of double-stranded DNA, or especially those based on intercalating compounds that allow the measurement of the length of synthesized polynucleotide molecules via fluorescence, fluorescence quenching or photometric measurements.

Particular preference is also given to variants in which the sample molecules to be measured are first bound to the cryogels containing the immobilized molecules having specific binding sites, followed by molecules which bind to the now bound sample molecules, for example antibodies which are for example conjugated with specific binder molecules such as streptavidin, avidin or preferably biotin, or are free, and subsequent binding of compounds (detection molecules) which bear fluorophores or other dyes and are specific for the molecules bound to the sample molecules, such as biotin or preferably streptavidin or avidin, or with appropriately labeled antibodies which specifically bind the heavy chains of the first-bound antibodies and which are bound to enzymes such as horseradish peroxidase or green fluorescent protein and can be detected by enzyme reaction or by fluorescent or photospectroscopic means, in other words, methods analogous to sandwich ELISA.

In a particularly efficient (and rapid) method, detection molecules (e.g., fluorescent detection antibody and optionally other necessary reagents) are initially charged at the start of the microfluidic channel (e.g., in the capillary). The test can subsequently be carried out in one step (see, for example, FIG. 2 and the description in the example). Mass transfer is achieved in this case preferably passively (e.g., capillary filling followed by continuous mass transfer by coupling to superabsorbent polymers.

### BRIEF DESCRIPTION OF THE DRAWINGS

Particularly preferred embodiments of the invention are also found in the description, the abstract and the claims, which are all incorporated here by reference into the description.

The figures show:

FIG. 1: Schematic representation of a capillary during the production process for a capillary-based device according to the invention. The upper part shows a capillary after it has been filled with different polymer solutions, which are separated from one another by air pockets. The lower picture shows the same capillary after freezing and exposure to UV radiation and washing, as described in the example.

FIG. 2: Schematic representation of a test setup for carrying out enrichment and binding of sample molecules when using a device according to the invention.

FIG. 3: Schematic representation of a selected capillary as microfluidic element according to the sandwich immunoassay carried out as per the example for the detection of analytes, together with (here exemplarily) fluorescence reader.

FIG. 4: Measured fluorescence intensities (=average gray value T−average gray value NC) for the IL-6 concentrations used in the example. For each concentration, two data points (two capillaries) are plotted. The blank measurement was carried out five times to ascertain the detection limit (LOD signal=Blank+3σ) via the calibration curves (dotted line, R²=0.997). LOD=26 pg/mL.

### DETAILED DESCRIPTION

The following example serves to illustrate the invention without limiting its scope, but also represents special embodiments of the invention.

The relevant test setup is demonstrated purely exemplarily by the figures described in detail, including with respect to the reference signs, hereinbelow (the relevant reference signs can also be used in the more general description and the claims in the case of the corresponding features):

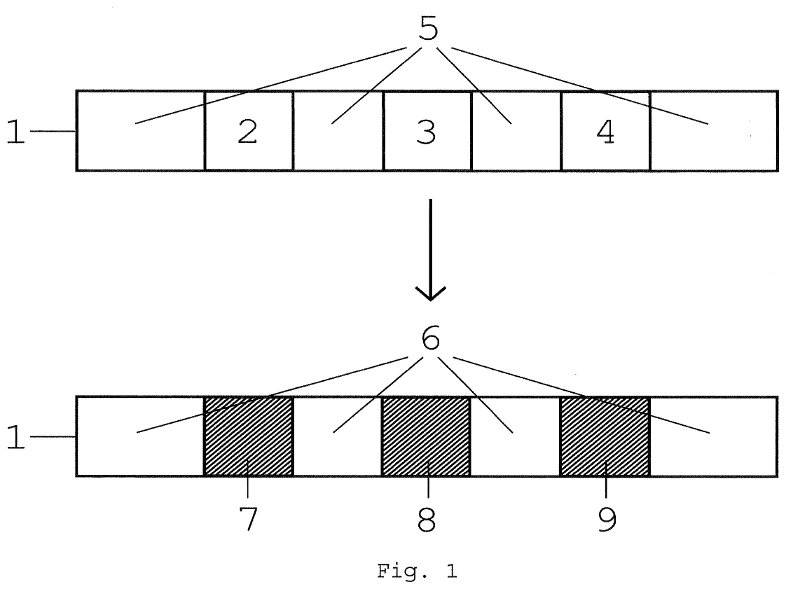

To demonstrate both the production process for and the bioanalytical applicability of the device according to the invention, commercial glass capillaries as an example of microfluidic elements 1 (Minicaps 5 μL, Hirschmann Laborgeräte GmbH & Co. KG, Hauptstraße 7-15, 74246 Eberstadt, Germany) having a diameter of 450 μm were first treated with a benzophenone-containing silane (triethoxy benzophenone silane, see O. Prucker, C. A. Naumann, J. Rühe, W. Knoll and C. W. Frank, J. Am. Chem. Soc., 1999, 121, 8766-8770) and subsequently filled, with the aid of a peristaltic pump (IPC ISMATEC from Cole-Parmer GmbH, Futtererstr. 16, 97877 Wertheim, Germany), with three different liquid compartments 2, 3 and 4, which were separated from one another in each case by an air pocket as separation substance 5 (see FIG. 1). The liquid compartments were:

1. 60 mg/L of a benzophenone-containing copolymer (PDMAA-5% MABP-2.5% SSNa, see M. Rendl et al., Langmuir, 2011, 27, 6116) dissolved in PBS (=NC) (liquid compartment 2 in FIG. 1)

2. NC+0.05 mg/mL of an antibody against human interleukin 6 (IL-6) (=T) (MAB206-100, R&D Systems, Bio-Techne GmbH, Borsigstraße 7a, 65205 Wiesbaden-Nordenstadt, Germany) (liquid compartment 3 in FIG. 1)

3. NC+0.01 mg/L biotinylated BSA (=PC) (A8549, Sigma-Aldrich Chemie GmbH, Munich, Germany) (liquid compartment 4 in FIG. 1).

In each case, 0.45 μL of liquid (A, B or C) or air as separation substance 5 were filled in.

(Note: The formula of the polymer used under 1. can be depicted as follows:

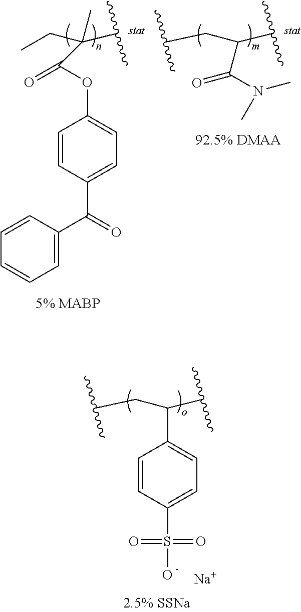

The distribution of the individual monomers in the polymer is random.)

The filled liquid compartments A, B, C were subsequently frozen by cooling the capillaries to −25° C. After freezing, the capillaries were exposed to UV light (365 nm) for 15 min (corresponding to an energy dose of around 30 J/cm²) (VL-UVA 135.M, 365 nm, 28 mW/cm², Vilber Lourmat, Germany) in order to photochemically excite the benzophenone groups. As a result, three processes take place simultaneously through nonspecific, free-radical reactions (C-H insertion reactions):
crosslinking of the polymer strands (development of the cryogel matrix)
attachment of the sample molecules (antibody and BSA) to the cryogel matrix
attachment of the cryogel matrix to the silanized glass capillary wall (spatial fixation of the constituents of the liquid compartments as compartments).

Cryogels produced by this method had pore sizes in the low μm range (typically 5-25 μm).

Resultant microfluidic elements 1 are depicted in FIG. 1, bottom. In said figure, 6 refers to the regions without molecules having a specific binding site (regions of the separation substance 5 in FIG. 1, top), containing in this case the wash buffer used for washing. 7, 8 and 9 refer to cryogel sections bound to the fluidic element 1, corresponding to the liquid compartments 2, 3, 4 stated above under A, B and C.

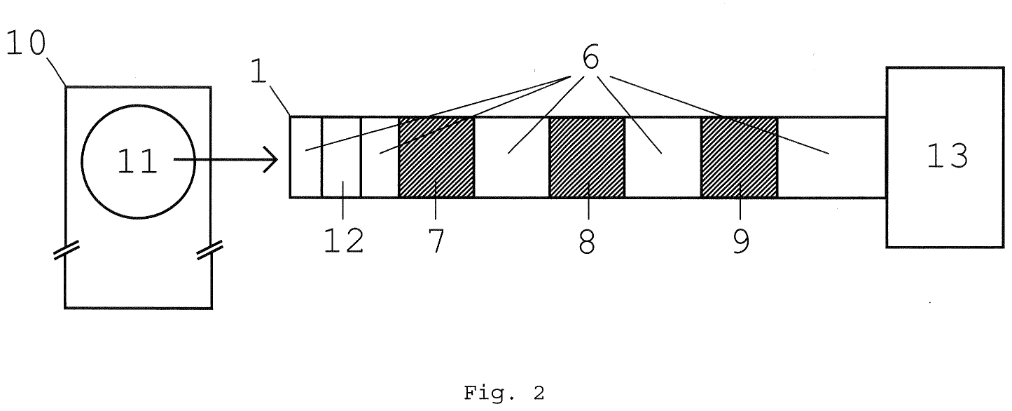

FIG. 2, besides the cryogel sections 7, 8 and 9, shows exemplarily part of the test setup, wherein further besides already described features from FIG. 1. A gaseous or, in this case, liquid sample containing analytes 11 from a microtiter plate 10 is drawn through the microfluidic element 1—in this case, by means of a pump device 13. In parallel, further (not depicted) such microfluidic elements 1 are fed from different wells having different liquid samples containing analytes 11—each microfluidic element 1 can, then, be connected to a, for example, peristaltic multichannel pump as pump device 13. Thereafter, the assay components, which are initially charged in a multi-well plate (microtiter plate), are drawn successively through the capillaries. (12 shows—unlike in the following example—a possible site for an alternatively possible initial charging of detection molecules).

In the example, the procedure is as follows: after the exposure, the capillaries (as microfluidic elements 1) were thawed and connected to a peristaltic 12-channel pump as pump device 13 (IPC ISMATEC from Cole-Parmer GmbH, Futtererstr. 16, 97877 Wertheim, Germany). The capillaries were washed for approx. 2 hours with PBS/0.1% BSA at an average flow rate of 2 μL/min. Thereafter, a classic sandwich immunoassay (with optical detection of a fluorescently labeled detection antibody) was carried out using various IL-6 standards (0 to 1000 pg/mL in PBS/0.1% BSA). To this end, the following liquid samples containing analytes 11 and reagents/solutions were pumped in steps through the capillaries for the indicated time:

1. IL-6 standards for 90 min

2. PBS/0.1% Tween for 10 min

3. Biotinylated detection antibody BAF206, R&D Systems, Bio-Techne GmbH, Borsigstraße 7a, 65205 Wiesbaden-Nordenstadt, Germany) (1 μg/mL in PBS/0.1% BSA) for 40 min

4. PBS/0.1% Tween for 10 min

5. Streptavidin-Cy5 (1 μg/mL in PBS/0.1% Tween) for 20 min (PA45001, GE Healthcare Europe GmbH, Oskar-Schlemmer-Str. 11, 80807 Munich, Germany)

6. PBS/0.1% Tween for 15 min.

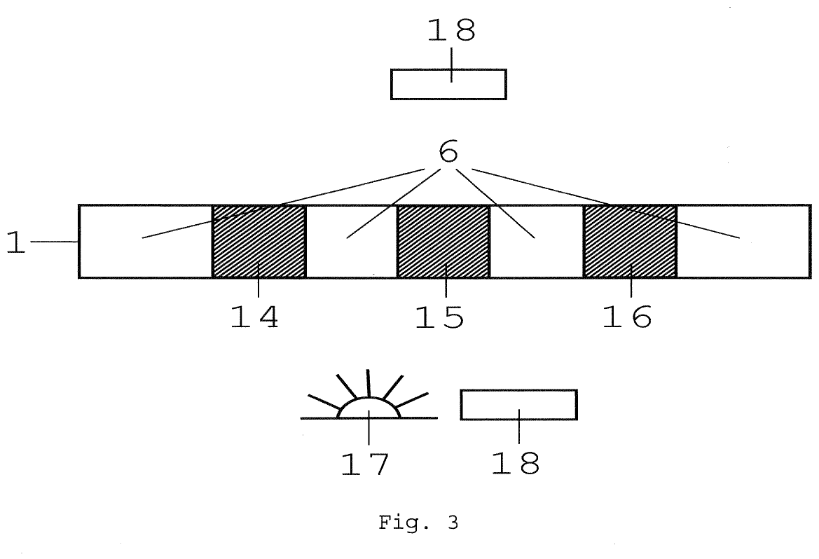

The sections 14, 15 and 16 in FIG. 3 that follow from the cryogel sections 7, 8, 9 shown in FIG. 1 and FIG. 2 have the correspondingly specifically bound sample molecules, to which biotinylated detection antibody and streptavidin-Cy5 are bound. Fluorescence is excited by a light source 17 and detected by an optical detector 18 (which can be arranged at any site, for example at the two positions shown).

In the specific example, the capillaries were analyzed in a commercial fluorescence reader (Fluorescent Array Imaging Reader, Sensovation AG, Markthallenstraße 5, 78315 Radolfzell, Germany).

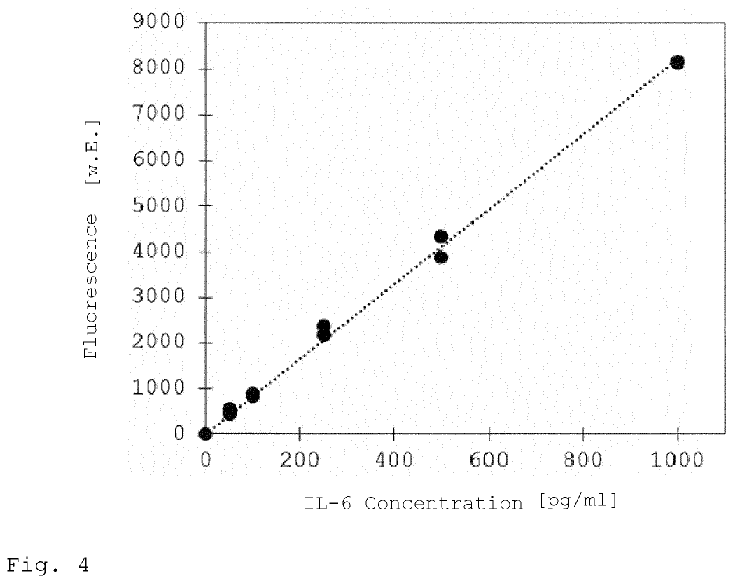

The results present with different amounts of IL-6 are shown in FIG. 4. The linearity allows a calibration, by which IL6 samples can then be quantified.

1. A device comprising a channel including a wall and having a sequence of compartments containing molecules having specific binding sites, said sequence of compartments is adapted for specific binding of analytes, porous cryogels chemically bonded to the wall, and the molecules having specific binding sites are bound to the porous cryogels as a support.

2. The device as claimed in claim 1, wherein the channel comprises a microchannel.

3. The device as claimed in claim 1, further comprising a microfluidic chip on which the channel is located.

4. The device as claimed in claim 1, wherein the channel comprises microfluidic elements.

5. The device as claimed in claim 4, wherein each said microfluidic element has multiple compartments, each comprising the molecules having specific binding sites, said molecules are bound to the porous cryogels in each case and are different for each compartment, and further comprising compartments without cryogels as separation regions.

6. The device as claimed in claim 1, further comprising means for generating pressure differences comprising at least one of a centrifuge, a centrifugal force-generating device, an external upstream or downstream pump system, an absorption material which supply or discharge liquids in a reproducible manner, or a microfluidic pump device.

7. The device as claimed in claim 1, wherein the channel comprises microfluidic channels configured for a flow generated solely or at least in part by capillary forces acting in the microfluidic channels.

8. The device as claimed in claim 1, wherein the cryogels comprise copolymers with functional groups including at least one of vinyl, epoxy or aldehyde groups or benzophenone groups including at least one of, polyurethane, epoxides reacted with polyamines or polyols, amino group-containing molecules bound by dialdehydes, prepolymers, polymers obtainable from ethylenically unsaturated molecules by free-radical chain reaction, azido group-containing monomers or polymers, anthraquinone-, or psoralen (derivative)-consisting or obtainable materials; or copolymers comprising benzophenone groups from/of a copolymer of the simplified formula 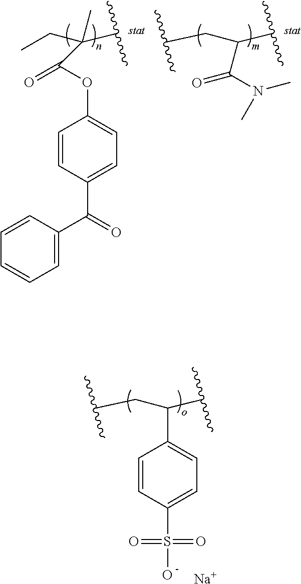
where “stat” stands for randomly arranged segments and means a random arrangement of the groups shown.

9. The device as claimed in claim 4, wherein the microfluidic elements are formed of glass or plastic which is transparent for visible or UV light.

10. The device as claimed in claim 1, wherein the channel is a microchannel having a smallest diameter transverse to a longitudinal direction across parts or an entire length of the microchannel of 1000 μm.

11. A process for producing a device as claimed in claim 1, comprising alternately, supplying volumes of initially charged solutions containing, firstly, precursor molecules of the porous cryogels as support and, secondly, the molecules having specific binding sites for the specific binding of analytes that are to be immobilized and are different for each volume, or the precursor molecules thereof, in sequence to the channel; cooling the filled device in order to freeze the solutions contained therein; and then carrying out reactions to develop a formation of the cryogels, a binding thereof to the wall of the channel and a binding of the molecules having specific binding sites that are to be immobilized or the precursor molecules thereof.

12. The process as claimed in claim 11, further comprising triggering the reactions by electromagnetic radiation.

13. The process of claim 11, wherein the channel comprises a microfluidic element.

14. A method of performing an assay using the device of claim 1, comprising conducting a liquid or gaseous sample containing sample molecules through the device and identifying bound molecules.

15. The method as claimed in claim 14, further comprising initially charging detection molecules at a start of the channel which comprises a microfluidic channel, and subsequently carrying out a test in one step, with mass transfer being passively.

16. The method of claim 15, wherein the passive mass transfer includes capillary filling followed by continuous mass transfer by coupling to superabsorbent polymers.

17. The device as claimed in claim 7, further comprising an absorbent material downstream of the microfluidic channels or placed at ends thereof.

18. The process of claim 11, further comprising, supplying, between the volumes containing the cryogel precursors and the molecules having specific binding sites that are to be immobilized or the precursor molecules thereof, precursor molecules of the cryogels without molecules to be immobilized or other liquid or gaseous separation substances in order to form a separation of the cryogels containing different ones of the molecules as the specific binding sites.

19. The process of claim 11, further comprising thawing and rinsing the cryogel.
